(19) United States
(12) Patent Application Publication
(10) Pub. No.: US 2023/0054371 A1
(43) Pub. Date: Feb. 23, 2023

(54) MEDICAL EVALUATION SYSTEMS AND METHODS USING ADD-ON MODULES

(71) Applicant: Analytics for Life Inc., Toronto (CA)

(72) Inventors: Ian Shadforth, Morrisville, NC (US); Abhinav Doomra, North York, CA (US); Ali Hussain, Mississauga, CA (US); Charlie Pham, LaSalle, CA (US); Murugathas Yuwaraj, Unionville, CA (US); Zhan Huan Zhou, Toronto (CA)

(21) Appl. No.: 17/891,547

(22) Filed: Aug. 19, 2022

Related U.S. Application Data
(60) Provisional application No. 63/234,772, filed on Aug. 19, 2021.

(51) Int. Cl. G16H 40/67; G16H 15/00; G16H 50/20

(52) U.S. Cl. CPC G16H 40/67; G16H 15/00; G16H 50/20

(57) ABSTRACT

A system is provided that receives a signal file that includes multiple biophysical signals obtained from a patient by a signal capture or recorder device. The biophysical signals are measured from one or more sensors or probes of the signal capture device. The system executes one or more add-on modules that is each configured to generate information relevant to the health of the patient. Such information may include a score that in some embodiments represents a probability that the patient has and/or will develop a particular medical condition. The information generated for a patient from the signal file for each add-on module are provided to a health care provider and may be used to assist the healthcare provider in diagnosing the patient with respect to one or more of the medical conditions.

### RELATED APPLICATION

This US application claims priority to, and the benefit of, U.S. Provisional Patent Application No. 63/234,772, filed Aug. 19, 2021, entitled “MEDICAL EVALUATION SYSTEMS AND METHODS USING ADD-ON MODULES,” which is incorporated by reference herein in its entirety.

### BACKGROUND

The design, development, manufacturing, marketing, and sale of regulated products such as medical devices, diagnostic products and services, and the like involve challenges not found in other fields. Significant scrutiny is paid by regulatory agencies in all aspects of these industries, requiring that manufacturers, distributors, sellers, and healthcare professional users follow a daunting labyrinth of laws, regulations, and other standards not found in other industries. Strict design control and quality systems, clinical and bench testing, complaint handling, post-market surveillance, and other requirements must be followed in addition to obtaining regulatory clearance to market such products and services. For these and other reasons, the time and cost to develop and bring a new medical device or diagnostic product/service onto the market can be significant. Other industries, such as transportation, food, pharmaceuticals, some consumer products, etc., also have these burdens to some degree.

Design and development paradigms that take these challenges into account can mitigate many of their negative impacts. In particular, approaches that simplify and improve designs, streamline and shorten development and testing cycles, reduce quality system impacts and lessen administrative and documentation workloads can result in products having greater reliability, security, transparency, maintainability, lower cost, etc. And in the case of medical devices and diagnostic products and services, such approaches ultimately can result in shortened regulatory clearance cycles, faster time to market, and better and more efficient healthcare outcomes. Systems and methods are described herein that exemplify such an approach in the context of a medical evaluation system.

### SUMMARY

A modular medical evaluation system is provided that includes a base system (that may optionally include a signal capture or recorder device) and, in other embodiments, one or more add-on modules. The medical evaluation system receives a signal file that includes biophysical signals obtained from a patient. The biophysical signals are obtained in some embodiments non-invasively from multiple sensors or probes communicatively (and in some embodiments physically) connected to a signal capture or recorder device. In other embodiments, minimally invasive or invasive sensors or probes may be utilized. The evaluation system can also include one or more add-on modules that is each configured to work in connection with the base system without any or any substantive changes to the software, firmware, and hardware of the base system (and documentation thereof) to generate information related to the health of the patient, including, for example, information related to the biophysical signals of the patient and/or information related to a particular condition, such as a disease state, of the patient. In some embodiments, this information can include, among other things, a score and/or other indicator(s) that represents a probability that the patient has or will develop one or more particular medical condition(s). This information is provided to a physician or other health care provider and may be used to assess the health and/or diagnose the patient with respect to one or more of the medical conditions, make treatment decisions, etc. In some embodiments, a signal capture or recorder device is considered part of the medical evaluation system; e.g., part of the medical evaluation system's base system component; in other embodiments, a signal capture or recorder device is separate from the medical evaluation system. Likewise, in some embodiments of the present disclosure, the medical evaluation system comprises simply the base system (with or without a signal capture or recorder device) that does not include, but that is capable of interfacing with, one or more add-on modules. The independence and compatibility of the base system compared to one or more add-on modules enable various advantages as disclosed herein. As such, various embodiments of the present disclosure can range from a base system alone with no add-on module to a base system together with one or any number of add-on modules.

Because of the modular nature of the medical evaluation system, its capabilities can be customized. For instance, the system's capabilities can be increased by including other add-on modules that are associated with, e.g., other conditions or disease states. In the context of the cardiovascular system, add-on modules that relate to coronary artery disease, heart failure in its various forms, arrhythmia, hypertension, etc., may be included in the system. Other configurations of the system may include add-on modules specific to related physiological systems, such as is the case for diseases such as pulmonary hypertension in its various forms that involve both the cardiovascular and respiratory systems. Other physiological systems such as neurological, endocrine, digestive, immune, musculoskeletal, etc., alone or in combination, can demand system configurations with add-on modules in any number (e.g., one to ten or more) that are optimized for the intended purpose. Add-on modules can not only provide information related to a specific disease state (e.g., a score indicating the likelihood or probability that the patient does or does not have coronary artery disease) but also information related to the state or function of a patient's body (e.g., a score indicating the likelihood or probability that a patient has elevated or non-elevated left ventricular end-diastolic pressure levels, indicators of ejection fraction, rhythm metrics, valve functionality, percentage of a coronary vessel that is blocked, etc.). Various add-on modules may use some or all of the collected biophysical signals as necessary to achieve their purpose.

In some embodiments, each add-on module uses the same signal files initially analyzed, processed, and stored by other components of the system described herein (e.g., the base system and/or a signal capture device). In some embodiments, the base system and/or signal capture device contains hardware, software, and/or firmware (and related documentation) that generally remains the same, regardless of which add-on module is utilized. These factors contribute to more efficient and faster development, documentation, regulatory review cycles, and time to market than otherwise possible. The modular architecture of embodiments of the system can simplify and generally improve designs, simplify and shorten development cycles, reduce quality system impacts, administrative and documentation workloads, and provide products with improved reliability, security and privacy, transparency, maintainability, etc. For example, the extension of the medical evaluation system as discussed herein to include one or more additional add-on module(s), or even completely new add-on modules, does not generally require modification to the software, firmware, and/or hardware—and documentation thereof—of the base system. Communication components and protocols designed into the base system and each of the add-on modules provide for seamless and secure transfer of data, commands, and other operational aspects of the system as add-on modules change, again without any change to the software, firmware, and/or hardware of the base system and/or capture device. In some embodiments, the addition of different add-on modules in various combinations may require modifications to or new configuration files to ensure they work with the base system and/or any capture device, if included. In other embodiments, the addition of different add-on modules in various combinations may not require any modification to such configuration files. In still other embodiments, no configuration files are necessary.

In the regulatory context, in particular, review and clearance cycles can be improved, simplified, and shortened by these aspects of the system's modular configuration. For example, a configuration of a medical evaluation system might comprise a capture device, a base system, and one add-on module. Each component may be independently assessed and reviewed for regulatory purposes, and then the system as a whole (i.e., all components taken together) can be similarly reviewed. However, once such a configuration is cleared by a regulatory authority, subsequent configurations with the same base system and/or capture device but having, e.g., one or more new add-on modules (with or without the add-on module present in the previously-cleared configuration of the evaluation system) can be reviewed with greater expediency as the regulator can focus on the new add-on module(s), how they work together with the base system and/or capture device, and their impact on the safety and efficacy of the new system due to the fact that rest of the system is identical or similar to (e.g., without any change or without any substantive change to the software, firmware and/or hardware and their accompanying documentation) the previously-approved medical evaluation system. This saves both the time and costs associated with regulatory clearance—not to mention the other benefits described herein—and allows for patients to more quickly benefit from the improved and/or increased range of capabilities provided by the system with the new add-on module(s).

In an embodiment, a medical evaluation system is provided. The system includes a base system; and one or more add-on modules. Each add-on module is associated with a different condition of a plurality of conditions. The base system is adapted to conduct one or more operations, including: receive a signal file that comprises a plurality of signals obtained from a patient; provide the signal file to at least one add-on module of the one or more add-on modules; receive a score from the at least one add-on module, wherein the score relates to the health of the patient; generate a report including the received score; and provide the generated report.

Embodiments may include some or all of the following features. The base system may include an analytical engine. The analytical engine may analyze the signal file for quality and other metrics. The score may represent a probability or likelihood that the patient has a condition associated with the at least one add-on module. Each add-on module may be adapted to: receive the signal file from the base system; extract one or more features from the signal file; generate the score using at least one of the features of the extracted one or more features and a model trained to generate the score; and provide the generated score to the base system. The model may include one or more of a linear model, a decision tree model, a random forest model, a support vector machine model, a neural network model, a deep neural network model, a convolutional neural network model, an adversarial neural network model, and a genetic algorithm model. The plurality of signals may include biophysical signals. The plurality of signals may include one or more ballistic signals and acoustic signals. The biophysical signals may include one or more biopotential signals and one or more photoplethysmographic signals. The biophysical signals may include one or more of ballistic signals and acoustic signals. The base system may be further adapted to: perform a quality assessment of the plurality of signals; and request a new signal file if the plurality of signals fails the quality assessment. The signal file may be received from a medical device. The base system may be part of a cloud-computing device. The base system may be approved for marketing by a regulatory authority, and at least one of the add-on modules may be received after the system was cleared for marketing by a regulatory agency or body. The base system may be further adapted to: provide the signal file to each add-on module of the subset of add-on modules; receive a score from each add-on module; and generate the report including each received score. The base system may be further adapted to: receive a selection of a subset of the one or more add-on modules; provide the signal file to each add-on module of the subset; receive a score from each add-on module of the subset; and generate the report including each received score. The base system may be further adapted to charge a fee to a user thereof to obtain the score for each add-on module in the subset. The base system may conduct the operations without any change to any software, firmware, or documentation thereto.

In an embodiment, a method is provided. The method includes: receiving a signal file by a computing system from a device, wherein the signal file comprises a plurality of signals obtained from a patient using a plurality of sensors associated with the device; extracting one or more features from the signal file by the computing system; selecting a model trained to output a score with respect to a condition using at least some of the features of the plurality of features; generating a score using at least some of the features of the extracted one or more features by the computing system, wherein the score relates to the health of the patient; and providing the generated score to the device by the computing system.

Embodiments may include some or all of the following features. The score may represent a probability or likelihood that the patient has the condition or a disease. The plurality of signals may include biophysical signals. The plurality of signals may include one or more of ballistic signals and/or acoustic signals. The biophysical signals may include one or more biopotential signals and/or one or more photoplethysmographic signals. The biophysical signals may include one or more of ballistic signals and/or acoustic signals. The method may further include: performing a quality assessment of the plurality of signals; and requesting a new signal file from the device if the plurality of signals fails the quality assessment. The device may include a medical device. The model may include one or more of a linear model, a decision tree model, a random forest model, a support vector machine model, and a neural network model, a deep neural network model, a convolutional neural network model, an adversarial neural network model, and a genetic algorithm model.

In an embodiment, a medical evaluation system. The medical evaluation system includes: a signal capture device, a base system, and one or more add-on modules. The signal capture device is adapted to: capture a plurality of signals from a patient; and generate a signal file that includes the plurality of signals. Each add-on module is associated with a different condition of a plurality of conditions. The base system is adapted to conduct one or more operations comprising: perform a quality assessment of the plurality of signals of the signal file; provide the signal file to at least one add-on module of the one or more add-on modules; receive a score from the at least one add-on module, wherein the score relates to the health of the patient; generate a report including the received score and information about the signal file; and provide the generated report.

Embodiments may include some or all of the following features. The base system comprises an analytical engine. The analytical engine may analyze the signal file for quality and other metrics. The score may represent a probability or likelihood that the patient has a condition associated with the at least one add-on module.

In an embodiment, a method is provided. The method includes: receiving a plurality of signals relating to a patient by a signal capture device; generating a signal file that includes the plurality of signals by the signal capture device; performing a quality assessment of the plurality of signals of the signal file by an analytical engine; providing the signal file to at least one add-on module of the one or more add-on modules by the analytical engine; receiving a score from the at least one add-on module, wherein the score represents a probability that the patient has a condition associated with the at least one add-on module; generating a report including the received score and information about the signal file; and providing the generated report.

In an embodiment, a method is provided. The method includes: receiving a plurality of signals relating to a patient; generating a signal file that includes the plurality of signals; performing a quality assessment of the plurality of signals of the signal file; generating a score that represents a probability that the patient has a condition; generating a report including the received score and information about the signal file; and providing the generated report.

Additional advantages of the invention will be set forth in part in the description which follows, and in part will be clear from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate modular medical evaluation systems and methods. Together with the description, the figures further serve to explain the principles of the systems and methods described herein and thereby enable a person skilled in the pertinent art to make and use the modular medical evaluation systems and methods.

FIG. 1A is an example environment for generating a report via a modular medical evaluation system based on signals taken from a patient and other patient data;

FIG. 1B is an illustration of exemplary components fora base system and any number of add-on modules that comprise an embodiment of a medical evaluation system that generates a report based on signals taken from a patient and other patient data.

FIG. 2 is an illustration of one or more components of an example medical evaluation system;

FIG. 3 is an illustration of a method for generating a report including scores for one or more conditions based on a received signal file;

FIG. 4 is an illustration of a method for generating a score by an add-on module; and

FIG. 5 shows an exemplary computing environment in which example embodiments and aspects disclosed herein may be implemented.

### DETAILED DESCRIPTION

Reference will now be made in detail to embodiments of the medical evaluation system and method with reference to the accompanying figures. The same reference numbers in different drawings may identify the same or similar elements.

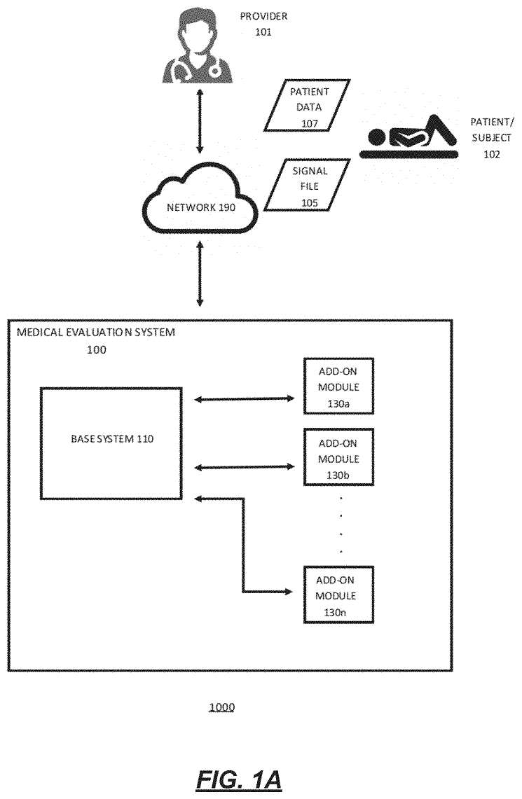

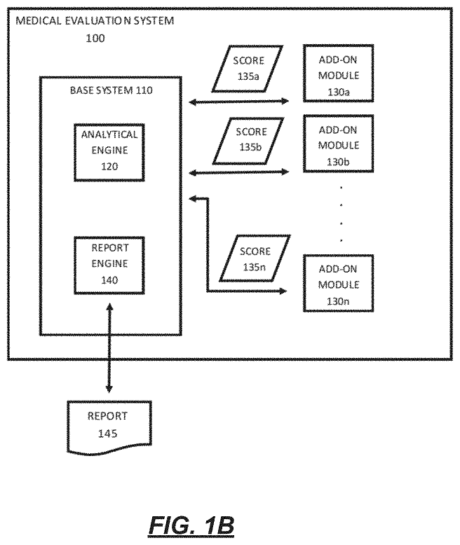

FIGS. 1A and 1B show an example environment 1000 (and a portion thereof, respectively) for generating one or more reports 145 as described herein based on signals taken from a patient and other patient data. As shown, environment 1000 includes a provider 101, a patient or subject 102, and a medical evaluation system 100 communicating through a network 190. In some embodiments, medical evaluation system 100 comprises a base system 110 alone. In other embodiments, medical evaluation system 100 comprises a base system 110 together with one or more add-on modules 130 (e.g., add-on modules 130a, 130b, and 130n). Evaluation system 100 can be a combination of hardware, software, and firmware components. For purposes of the present disclosure, reference to element 102 as “patient” means a patient and/or a subject of a clinical study or trial.

Environment 1000 can include, e.g., a particular operational environment such as a cloud-based server environment present in one geographical location or in multiple geographic locations for purposes of redundancy, reliability, security, optimal service and performance time, cost, etc. Environment 1000 can also include, e.g., a particular jurisdictional regime, e.g., the United States, Canada, European Union (or any country therein), China, Japan, etc. in each of which may exist a specific or unique regulatory, operational, security, and/or marketing paradigm.

Network 190 may include a combination of private and public networks such as the internet, mobile phone networks, wireless networks, and the like.

While only one provider 101, patient 102, and medical evaluation system 100 are shown in FIG. 1A, it is for illustrative purposes only: it is contemplated that for a given instance of evaluation system 100, there may be multiple providers 101 (and in turn, each of which may be responsible for one or more patients 102) and evaluation systems 100, in various combinations, in environment 1000. Other embodiments include multiple environments.

Each embodiment of evaluation system 100 may be configured to handle one or any number of patients in its operational capacity. In addition, the evaluation system 100 may be replicated and/or customized as operational, regulatory, marketing, cost, and other considerations dictate.

Evaluation system 100 is capable of receiving and processing biophysical signals of a patient 102. In some embodiments, evaluation system 100 includes a signal capture or recorder device (not shown), which can be reusable. In other embodiments, evaluation system 100 does not include a signal capture or recorder device. Evaluation system 100 may receive biophysical signals from a signal capture device, directly from one or more sensors, from a wearable device (e.g., smartwatch, clothing), or via other means, such as, e.g., from a file residing on a portable storage device, via a networked storage device (e.g., data residing in the electronic medical record of patient 102), etc.

In a method of use, a component of evaluation system 100 may receive a signal file 105 for a patient 102 from, e.g., a signal capture or recorder device (not shown) via a provider 101. Provider 101 may be a healthcare provider such as a physician, nurse, or medical technician. In some embodiments, a family member, friend, or even the patient themself can substitute for provider 101. In some embodiments, the signal file 105 may be generated by a signal capture or recorder device as described above that obtains signal data (e.g., biophysical signals) from the patient 102 through single or multiple probes or sensors in various combinations along with some embodiments patient-specific data or metadata. Depending on the embodiment, the probes or sensors may be non-invasively connected to the body of the patient (e.g., via placement of electrodes to acquire patient cardiac biopotential signals and/or a hemodynamic sensor attached to a finger, earlobe, etc. of the patient; an accelerometer attached to the skin of the patient, etc.). Such probes or sensors may also be invasively or minimally invasively attached to the patient. In other instances, probes or sensors capable of collecting biophysical signals of the patient for the signal file may be non-invasive and non-contact, such as in the case where passive thermometers, scanners, cameras, etc., are used).

A signal capture device may collect biophysical signals generated by the probes or sensors and may include such signals and/or related data in signal file 105. Example biophysical signals that may be collected include photoplethysmographic signals, biopotential signals, ballistocardiographic signals, and chemical signals. Other types of signals may be collected. Depending on the embodiment, signal file 105 may include signals collected instantaneously and/or over a predetermined amount of time (e.g., about ten seconds, about one minute, about three minutes, about five minutes, about twenty minutes, etc.). Signal recorder or capture device can include hardware, firmware, and software for communicating the captured biophysical signals with or without other information to other components—such as base system 110—of evaluation system 100. In one embodiment, signal capture device 102 includes a seven-channel lead set and a photoplethysmogram (PPG) sensor attachable (permanently or detachably) thereto, along with a user interface (e.g., (touch) screen, keyboard, speaker, microphone, camera/scanner, biometric sensor, etc. and combinations thereof) that allows a healthcare provider or other user 101 to enter patient-specific data or metadata 107 therein.

The patient data 107 may include additional information about the patient that may be useful for the patient's medical evaluation, such as height, weight, age, sex, body mass index (“BMI”), the medical history of the patient (including, e.g., symptom information, the presence or absence of certain diseases such as, e.g., diabetes), information relating to any medications that are currently or have been taken by the patient, results of any recent medical tests, such as blood tests, additional demographic information, etc. Other information may be included in patient data 107. Signal recorder or capture device can, in some embodiments, synchronously acquire patient signals through such sensors and securely transmit such signals in one or more signal files 105, along in some cases with patient-specific data 107, to a data repository that in such embodiments is part of base system 110 comprising medical evaluation system 100.

Medical evaluation system 100 may receive signal file 105 and/or patient data 107 and, as will be described further below, may use some or all of the data in the signal file 105 and/or patient data 107 to generate useful information relative to that patient. For example, such information may include one or more scores 135 related to each of one or more conditions. The conditions may include medical conditions, such as an illness or disease. In one embodiment, the higher the generated score 135 for a medical condition, the greater the likelihood or probability that the patient who has been evaluated by evaluation system 100 has the condition or is at risk of developing the condition. Score 135 can, along with other information obtained by provider 101 (via, e.g., physical examination of the patient, etc.), assist provider 101 in assessing or evaluating the medical status of patient 102, including diagnosing one or more conditions, determining that patient 102 does not have one or more conditions, deciding on whether to administer further tests and/or to embark on a treatment course (e.g., surgery, medication, nutritional/exercise/lifestyle changes), follow-up visits, etc. In other embodiments, a higher score 135 may indicate a lower correlation to a given condition. Other formats for providing such information in addition or as alternatives to a numerical score 135, such as a visual representation of such a likelihood, an anatomic localization of or relating to the condition, a written assessment, etc., are contemplated.

In other embodiments, score 135 is not provided, but other information useful for a health care provider in making care decisions for that patient is provided. In still other embodiments, score 135 is provided with other such information. Any combination of information, with or without score 135, may be provided. Depending on the embodiment, evaluation system 100 may generate each score 135 using a model that was trained to generate the score 135 based on signals and/or medical data taken from patients that were known to either have or not have the particular condition.

Medical evaluation system 100 can collect the one or more generated scores 135 and can use the score(s) 135 to generate a report 145 specific to patient 102. Evaluation system 100 may provide the report 145 to the patient's provider 101 or one or more other individuals; in some instances, report 145 may be sent directly to an electronic medical records system. Report 145 may include some or all of the generated scores 135 and an indication of the condition associated with each score. Depending on the embodiment, some of the signals of the signal file 105 and/or the patient data 107 that was used to generate the scores 135, including visualizations thereof, may be included in the report 145 along with any amount of other information that may be useful to provider 101 in assessing or evaluating the health of the patient.

FIG. 1A depicts a high-level structural view of evaluation system 100 in environment 1000 that highlights its advantageous modular aspects. At its core, medical evaluation system 100 comprises a base system 110 capable of operating in conjunction with one or more add-on modules 130a, 130b, . . . 130n. The architecture depicted in FIG. 1A affords independence of the software, firmware hardware, and associated documentation between base system 110 from that of any add-on module, directly resulting in the advantages discussed herein.

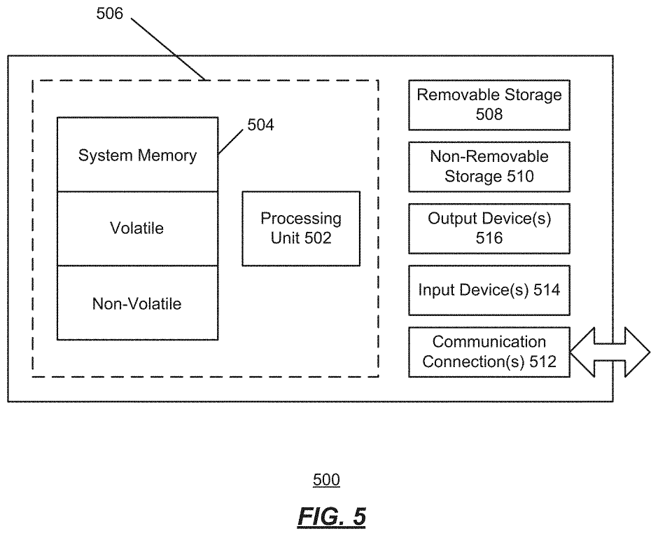

FIGS. 1A and 1B show base system 110 being compatible with, and capable of ready interfacing and use with, any number of n add-on modules 130 as may be desired. Computing resources, for example, may be readily scaled in base system 110 to accommodate the need for increased processing and data storage needs as the number of add-on modules for a given base system is increased without changing the base system's substantive software, firmware, and/or hardware infrastructure. Each component of evaluation system 100 may be implemented together or separately using one or more general-purpose computing devices such as the computing device 500 illustrated with respect to FIG. 5.

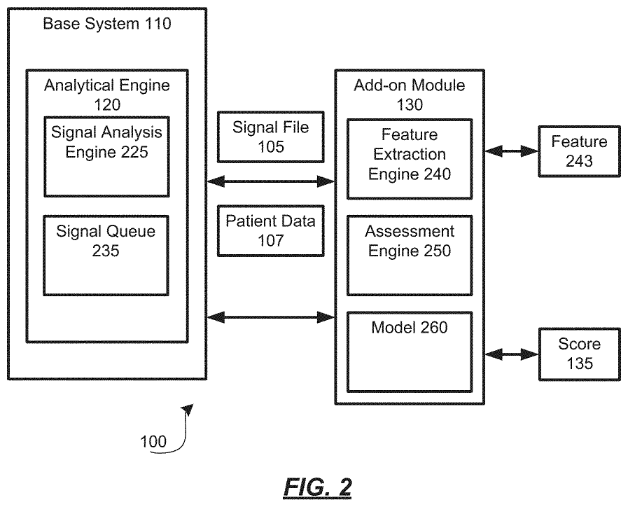

As shown in FIG. 1B, base system 110 may include an analytical engine 120 (which itself may include various components as discussed in connection with FIG. 2), and a report engine 140. Each of the one or more add-on modules 130 may include various components, as will be discussed in connection with FIG. 2. More or fewer components may be supported.

Medical evaluation system 100 may receive the signal file 105 and/or patient data 107 through, e.g., a networked signal recorder or capture device or by other means as described herein. One or more user interfaces on or associated with a signal capture or other device (e.g., a graphical user interface, keyboard, audible system (microphone and/or speaker), etc., individually or in various combinations) may be used by provider 101 or others to perform a variety of tasks such as entering information about the patient 102 (e.g., patient data 107), controlling and/or communicating with the signal capture device or other devices. For example, in one use scenario, provider 101 can enter patient data 107 into a signal recorder or capture device via its user interface; in turn, the signal capture device automatically or as commanded by provider 101 sends such data 107 to evaluation system 100 via network 190.

After preparing and connecting the patient to the various sensors and/or probes (or cases of non-contact sensors and/or probes, preparing for data collection as required by the specific sensor and/or probe type), provider 101 may indicate that they are ready to begin collecting sensor data by pressing a button or activating another user-interface element in the signal recorder device (e.g., voice command). Signal capture device 102 will begin (synchronously, in some embodiments) collecting the patient's biophysical signals. Upon completion of data acquisition, components of the signal recorder device prepare the acquired data in the form of signal file 105 for transfer, along with patient data 107 and/or other metadata (e.g., timestamp, software version number) in one or more data packets to a repository that is part of evaluation system 100 via network 190 through, e.g., a Wi-Fi or cellular/mobile (e.g., LTE, 5G, 6G, etc.) connection. In some embodiments, such a repository is a cloud-based storage device or is otherwise in a physical location apart from the signal recorder device. This repository may store signal file 105, patient data 107, and other metadata. After evaluation system 100 has analyzed and processed these data, as will be described in more detail below, provider 101 may use a portal, such as a web-based application or software running on a client computer, tablet, mobile device, etc. to view the one or more reports 145 generated for the patient 102 whose signals were so analyzed and processed.

Depending on the embodiment, rather than, or in addition to, a portal, medical evaluation system 100 may expose an application programing interface (“API”) through which provider 101 may interact with medical evaluation system 100. Provider 101 may then use the API to download or import data from evaluation system 100 (e.g., signal file 105, patient data 107, and/or report 145) into an application of their choosing (e.g., word-processing application, spreadsheet application, a database such as a medical records database, document management system, etc.).

Continuing to FIG. 2, details of the base system 110's analytical engine 120 and of a single exemplary add-on module 130 are shown. Multiple add-on modules 130 may be included.

In base system 110, analytical engine 120 may include one or more components, including a signal analysis engine 225 and a signal queue 235. More or fewer components may be supported. Depending on the embodiment, one or more instances of the analytical engine 120 may be executed in a cloud computing environment, for example.

Signal analysis engine 225 may receive a signal file 105 via a signal recorder device or other means as described herein and may, in some embodiments, perform a signal quality assessment operation (“SQA”). Depending on the embodiment, the SQA may be one or a series of tests that are performed on some or all of the signals of signal file 105 to determine if the signals in signal file 105 are of sufficient quality and/or duration for use by one or more of the add-on modules 130. Depending on the embodiment, the particular requirements (e.g., appropriate length or number of data points, specific quality metrics) of the signals may be universal or specific to each type of signal received by engine 225; they may also be specific to the add-on modules 130 that are to analyze the signals in a later stage. For example, quality metrics for electrical signals may differ from quality metrics for hemodynamic signals. And signals from other sensors or probes, such as ballistocardiographic sensors, may require even different quality metrics. Some quality metrics may be common to two or more signal types (i.e., based on the sensor or probe that collects such signals); in other embodiments, the metrics may be mutually exclusive. And in the case of add-on modules 130, some may require higher quality, different types, configurations, and/or longer-duration signals that make up a given signal file 105 than other add-on modules 130. The particular requirements of the SQA may in some embodiments be set by a user or administrator; in other embodiments, such SQA requirements are a fixed aspect of evaluation system 100.

When a new signal file 105 is received by analytical engine 120, signal analysis engine 225 may perform an SQA. If the SQA is not successful (e.g., the signals in the file 105 are not of sufficient length or quality), signal analysis engine 225 may request a new signal file 105 from provider 101. The request may be displayed to provider 101 in the portal or via a user interface in hardware running application software (e.g., via a user interface in a signal capture device) and/or other application that was used to submit signal file 105 and may indicate the particular reason or reasons that the original signal file 105 did not pass the SQA.

If signal file 105 passes the SQA, signal analysis engine 225 may store the results of the SQA as part of signal file 105 and may place signal file 105 into a signal queue 235. Signal queue 235 may store signal files 105 until they can be processed by one or more add-on modules 130. Depending on the embodiment, after placing signal file 105, signal analysis engine 225 may message one or more add-on modules 130 to indicate that the signal file is available for processing.

In some embodiments, signal analysis engine 225 may further generate one or more visualizations of the signals present in signal file 105 after the SQA is complete. The visualizations may be stored, in some embodiments, by the signal analysis engine 225 with the signal file 105. Analytical engine 120, including signal analysis engine 225 and signal queue 235 can collectively be considered the base system portion whose software, firmware, and hardware—and related documentation - are independent of that of any add-on module 130.

As described above, embodiments of evaluation system 100 may include no add-on module, one add-on module 130, or more than one add-on module 130. Each add-on module 130 can interface with base system 110, its analytical engine 120 and associated components, typically but not necessarily via a data transfer API or similar mechanism. As such, FIG. 2 shows a single add-on module 130 as an example of one configuration of an embodiment of evaluation system 100. Medical evaluation system 100 may also comprise any number (n) of add-on modules 130 that are different than module 130 shown in FIG. 2. The software, firmware, and any hardware associated with each of the one or more add-on modules 130 (and their documentation) may be completely or largely independent of the software, firmware, and/or hardware of base system 110, including analytical engine 120. Evaluation system 100 is designed in a modular way such that if one or more add-on modules 130 are included as part of evaluation system 100, each of whichever number of modules 130 are utilized, all integrate with base system 110 so that evaluation system 100 can realize its potential. In this way, medical evaluation system 100, when using one or more add-on modules 130, can utilize the same signals and/or patient data to assess for the presence of one or more disease states, conditions, etc. (e.g., one for each add-on module 130). In some embodiments, one or more APIs, such as a data transfer API residing in base system 110 can facilitate the secure access to, and transfer of data between base system 110 and each add-on module, as well as between the components within base system 110 and if present, a signal capture recorder or device. Use of a DTAPI or other API structure can result in increased control over the operations of the evaluation system 100, for example, in the security and data privacy contexts, and can facilitate the advantages the modularity of system 100 affords.

Each add-on module 130 can be configured to generate and output information useful to provider 101 for assessing the health of the patient, including, for example, a score 135 and/or other information as described herein. The information, including, for example, score 135, may be determined by the add-on module 130 using some or all of the signals of the signal file 105 and some or all of the patient data 107. Example conditions may be medical conditions or diseases and may include medical conditions such as elevated LVEDP, an indicator of heart failure, and diseases such as coronary arterial disease, pulmonary hypertension, etc. Other medical conditions and diseases or indicators of them may be included.

In one embodiment, evaluation system 100 includes a single base system 110 alone.

In another embodiment, such as that shown in FIG. 2, evaluation system 100 includes a single base system 110 together with a single add-on module 130, such as an add-on module 130 to indicate the likelihood that a patient has elevated left ventricular end-diastolic pressure (LVEDP).

In another embodiment, evaluation system 100 includes a single base system 110 together with a single add-on module 130, such as an add-on module 130 to indicate the likelihood that a patient has coronary artery disease (CAD).

In another embodiment, evaluation system 100 includes a single base system 110 together with a single add-on module 130, such as an add-on module to indicate the likelihood that a patient has elevated mean pulmonary arterial pressure (mPAP).

In another embodiment, evaluation system 100 includes a single base system 110 together with two add-on modules 130, such as an add-on module 130 to indicate the likelihood that a patient has elevated left ventricular end-diastolic pressure (LVEDP) and another add-on module 130 to indicate the likelihood that a patient has coronary artery disease (CAD).

In another embodiment, evaluation system 100 includes a single base system 110 together with two add-on modules, such as an add-on module 130 to indicate the likelihood that a patient has an elevated mean pulmonary arterial pressure (mPAP) and another add-on module 130 to indicate the likelihood that a patient has coronary artery disease (CAD).

In another embodiment, evaluation system 100 includes a single base system 110 together with two add-on modules 130, such as an add-on module 130 to indicate the likelihood that a patient has an elevated left ventricular end-diastolic pressure (LVEDP) and another add-on module 130 to indicate the likelihood that a patient has an elevated mean pulmonary arterial pressure (mPAP).

In yet another embodiment, evaluation system 100 includes a single base system 110 together with three add-on modules, such as an add-on module 130 to indicate the likelihood that a patient has an elevated left ventricular end-diastolic pressure (LVEDP), another add-on module 130 to indicate the likelihood that a patient has coronary artery disease (CAD), and a third add-on module 130 to indicate the likelihood that a patent has an elevated mean pulmonary arterial pressure (mPAP).

In yet still a further embodiment, evaluation system 100 includes a single base system 110 together with any number of add-on modules 130, with each add-on module 130 directed to a specific disease state, condition, etc., such as those described above and otherwise. Such disease state or condition may be related to any physiological system, such as cardiovascular, respiratory, neurological, endocrine, digestive, immune, musculoskeletal, etc., and combinations thereof.

In all of the aforementioned exemplary embodiments disclosed above and elsewhere herein, evaluation system 100 may also include a signal recorder or capture device.

It should be understood, then, that various embodiments of medical evaluation system 100 capable of operating in environment 1000 can comprise base system 110 together with any number of add-on modules 130 (e.g., zero, one, two, three, ten, twenty, one hundred, five hundred, etc.).

Moreover, the software, firmware and hardware of base system 110 can remain exactly or basically the same, with little or no modifications thereto necessary, and still successfully operate in concert with each add-on module 130 to achieve the purposes of medical evaluation system 100. As such, base system 110 alone (e.g., without any add-on module 130) is a valuable technological advance that, as a foundational part of evaluation system 100, provides a significant advantage in the design/development, reliability, regulatory, security, marketing, and other realms as discussed herein.

As shown in FIG. 2, each add-on module 130 may include one or more components, including a feature extraction engine 240, an assessment engine 250, and a model 260. More or fewer components may be supported. Depending on the embodiment, each add-on module 130 may be executed in the cloud computing environment, for example. Each add-on module 130 may be isolated from one another, and the analytical engine 120, using a container such as a docker. Various add-on modules 130 may take on different configurations while still being part of evaluation system 100 in environment 1000.

Feature extraction engine 240 may retrieve a signal file 105 from the queue 235 and may extract one or more features 243 from signal file 105. The particular features 243 extracted from signal file 105 may include those features 243 that are considered by the model 260 of add-on module 130 and may depend on the particular condition or system that is the focus of the add-on module 130. Example features in the cardiovascular and respiratory context include, but are not limited to, depolarization or repolarization wave propagation associated features, depolarization wave propagation deviation associated features, cycle variability associated features, dynamical system associated features, cardiac waveform topologic features, PPG waveform topologic features, wavelet PPG features, features targeting left ventricular hypertrophy, particularly linear of such features, repolarization features, cardiac or PPG signal power spectral density associated features, cardiac or PPG or LVEDP signal visual associated features, and in some embodiments predictability features. In an embodiment of an add-on module 130 related to elevated LVEDP, feature extraction engine 240 may extract 446 features from 18 feature families. More or fewer features may be extracted depending on the particular add-on module.

In some embodiments, where feature extraction engine 240 cannot successfully extract a particular feature 243 from the signal file 105, feature extraction engine 240 may impute a value for the particular feature. For example, feature extraction engine 240 may use a median value for the feature based on values of the feature extracted from previous signal files 105 (i.e., historical feature values).

Feature extraction engine 240 may further extract one or more features 243 from the patient data 107 in any combination. Example features include, but are not limited to, BMI, age, height, weight, and gender.

Assessment engine 250 may use some or all of the extracted features 243 and model 260 to generate the information, including for example score 135. In some embodiments, model 260 may have been trained to generate the information, including in some embodiments score 135, using extracted features 243 associated with individuals known to have the condition and extracted features 243 associated with individuals known not to have the condition. Example models 260 include, but are not limited to, various classifier models such as linear models (e.g., Elastic Net), decision tree models (XGB Classifier), random forest models, support vector machine models, neural network models, deep neural network models, convolutional neural network models, adversarial neural network models, genetic algorithm models, etc.

Secure access, communications, and data transfer between base system 110 and any number of add-on modules 130, as well as among various components of each, may be achieved in any number of ways. For example, native cloud-based functionality/features may accomplish these functions. In other embodiments, for example, one or more APIs, such as a data transfer API (DTAPI) may accomplish these functions to provide, e.g., greater control over security functionality.

Returning to FIG. 1B, report engine 140 may receive information, including for example one or more scores 135 (e.g., the scores 135a, 135b, and 135n), from some or all of the add-on modules 130. Report engine 140 may then construct a report 145 using the received information, including for example one or more scores 135. In some embodiments, report 145 may include each score 135 and along with text that describes the condition to which score 135 relates. Report 145 may further include context information with each score that explains the relative risk or probability of the condition to which score 135 relates (e.g., in any graphical, tabular, textual, visual, etc., formats in any combination).

In addition, report engine 140 may also include in report 145 certain statistical information, such as a confidence interval, along with each score 135 that represents the confidence that the score 135 is correct. Other statistical information can include, e.g., information to accompany the confidence interval, score-specific information such as AUC-ROC curve data (e.g., score and/or threshold statistics such as sensitivity and specificity values), frequency distribution plots, and information, etc.).

Additional information related to the acquired biophysical information of the patient, such as any visualization data generated by signal analysis engine 225 (e.g., VCG plots of electrical data, amplitude-time plots of hemodynamic data, voltage-acceleration, amplitude-time plots of ballistocardiographic data, etc.), may be included in report 145. The contents of report 145 (e.g., data and visualizations) related to scores 135 (e.g., scores 135, context information, and statistical information) may be generated by add-on module 130 and provided to report engine 140. The contents of report 145 related to the acquired biophysical information of the patient may be generated by analytical engine 120 and/or report engine 140.

Report engine 140 may store the generated report 145 and may provide the report 145 to provider 101. Report engine 140 may provide report 145 to provider 101 through a portal that is used by provider 101 to view data related to their patients. Report engine 140 may generate the portal, and provider 101 may log into the portal and may select a patient whose report 145 they would like to access.

In some embodiments, the report 145 may include the scores 135 generated by all of the add-on modules 130 associated with report engine 140. For example, the provider 101 may receive a report 145 that includes scores 135 for every condition associated with an add-on module 130 supported by evaluation system 100. In other embodiments, the report 145 may only include scores 135 generated by the add-on modules 130 for conditions or groups of conditions that are selected by provider 101. For example, provider 101, using the portal or API, may select each condition that they would like to include in the report 145.

In embodiments where report 145 only includes information and, e.g., scores 135, for selected conditions, only the add-on modules 130 associated with a selected condition may generate a score 135 based on the provided signal file 105 and/or patient data 107. Medical evaluation system 100 may save the signal file 105 and/or patient data 107 so that if provider 101 later wants to add one or more conditions to report 145 (or new conditions as corresponding add-on modules 130 become available) the saved signal file 105 and/or patient data 107 can be used.

Alternatively or additionally, regardless of which conditions are selected by provider 101, each add-on module 130 may generate a score 135, but only the scores 135 associated with the selected conditions may be included in report 145. Scores 135 associated with non-selected conditions may be stored by evaluation system 100 should provider 101 later decide to include them in report 145.

As described above, each of the one or more add-on modules 130, each of which includes its own feature extraction engine 240, assessment engine 250, and model 260, may be implemented as software modules that can be selectively added or removed from evaluation system 100. This may allow evaluation system 100 to be continuously improved or periodically updated by adding new add-on modules 130 for previously unserved conditions and/or by replacing or modifying existing add-on modules 130 as their associated models 260 are improved upon—all without having to modify base system 110.

Another advantage of the modular base system-add-on module architecture of evaluation system 100 as described herein is related to obtaining marketing clearance or authorization by regulatory agencies such as the U.S. Food and Drug Administration (FDA), Health Canada, the European Medicines Agency and national competent authorities, etc. Initially, evaluation system 100 (separately or together with the signal recorder device that generates the signal file 105) may receive marketing clearance from the applicable government agency such as the FDA or an authorized entity such as a notified body. Over time, as new add-on modules 130 are developed for use in evaluation system 100, review and clearance timeframes for new add-on modules may be streamlined as they are intended and designed for use with a cleared evaluation system 100, including the portion of evaluation system 100 (e.g., a base system) that is independent of previously-approved add-on modules 130. In other words, because the only changes made to evaluation system 100 is to the new add-on module or modules 130, the reviewing agency or notified body should be able to more quickly review and clear the improved system 100 for marketing.

Another advantage of the modular base system-add-on module architecture of evaluation system 100 as described herein includes improved design, development, and testing timelines. Because the overall design of evaluation system 100 is not changed and a base system is available, e.g., for many visualization and data management functionalities, researchers can focus only on the creation of the new add-on modules 130 without considering how the various signals are collected and managed for each patient 102. This may result in the faster (and more cost-effective) development of new and improved add-on modules 130 when compared with conventional medical device development paradigms.

Yet a further advantage of the modular base system-add-on module architecture of evaluation system 100 as described herein is the overall reduction in the documentation that is needed when new features are added. Because changes to evaluation system 100 are limited to add-on modules 130, when changes are made to evaluation system 100 most of the documentation from the previous version may be reused without any significant changes.

Another advantage of the modular base system-add-on module architecture of evaluation system 100 as described herein includes higher operational efficiency, such as improved reliability. Because each add-on module 130 is executed separately in its own container, if there is a problem with the operation of a particular add-on module 130 it may be more readily and in some cases quickly detected—and if necessary disabled—so that the problem can be isolated so as to not affect the operation of the other add-on modules 130, or the overall evaluation system 100 itself. This in turn allows for less system downtime, increased leverage in commercial negotiations, lower costs, and reputational gains with third parties, such as customers and competitors.

Yet still another advantage of the modular base system-add-on module architecture of evaluation system 100 as described herein is in the security realm. With any system operating in a network environment 190 such as those described herein, care must be taken to ensure sufficient data and operational security are built in, especially when sensitive patient data may be involved. The modular nature of evaluation system 100 allows security measures to be built into each of the add-on modules 130 and into base system 110, and of course, with the communication protocols among the various components of each as well as between them. Multiple redundancies of security protocols, along with hosting of data and software infrastructure at multiple sites, are more readily implemented with a modular design. For example, a security breach in one component of evaluation system 100 has a higher likelihood of being identified, isolated, and remedied more quickly, without the breach affecting other components, due to the relative independence and customized security features that can be built into each component of evaluation system.

Another advantage of the modular base system-add-on module architecture of evaluation system 100 as described herein lies in the ability to more quickly introduce new add-on modules to address unmet clinical and business needs. Streamlined design/development, testing, and regulatory timelines along with enhanced operational and security performance translates into a significant advantage with providers 101, payors, the government, and other customers.

Any or all of these above-noted advantages can be realized for systems such as those described herein in industries outside the medical device and diagnostic fields, resulting in faster-to-market and more profitable business models—even those that are not as heavily regulated.

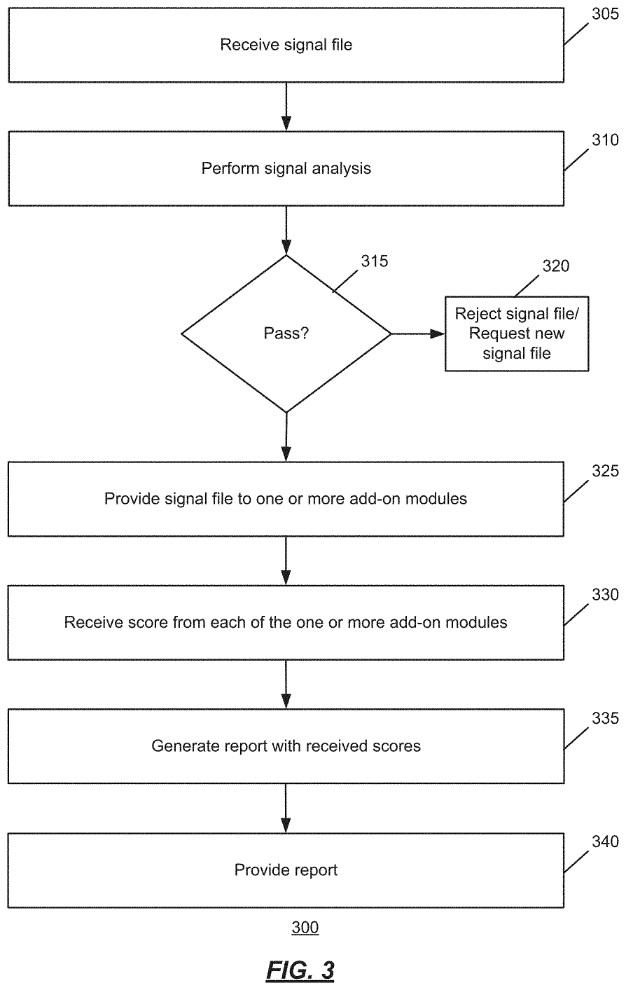

FIG. 3 is an illustration of a method 300 for generating a report including scores for one or more conditions based on a received signal file. The method 300 may be implemented by the base system 110 of medical evaluation system 100.

At 305, a signal file is received. Signal file 105 may be received from a provider 101 by evaluation system 100. Signal file 105 may include a plurality of biophysical signals captured from a patient by a signal capture device 102 via one or more probes and/or sensors. The biophysical signals may include photoplethysmographic signals, biopotential signals, and/or ballistocardiographic signals, among others. Evaluation system 100 may receive signal file 105 from the signal capture or recorder device 102 through a portal or API provided by evaluation system 100. In some embodiments, evaluation system 100 may also receive patient data 107 associated with the patient that may include information such as the gender, height, weight, age, and/or BMI of patient 102. Other information may be included. The received signal file 105 and patient data 107 may be stored in an account associated with provider 101 and/or patient 102.

At 310, signal analysis is performed. The signal analysis may be performed by analytical engine 120 of system 100. The signal analysis may include an SQA and may determine if the signals in signal file 105 are of sufficient quality and/or duration to be used by one or more add-on modules 130. Depending on the embodiment, the SQA may output one or more metrics related to the quality and/or duration of each of the signals in signal file 105. The results of the SQA may be stored in the account associated with the provider 101 and/or patient.

At 315, whether the signal file passed the signal analysis is determined. The determination may be made by analytical engine 120 of evaluation system 100. Depending on the embodiment, signal file 105 may pass the signal analysis when, in the context of an SQA, the determined quality and/or length of some or all of the signals is greater than one or more quality and/or length thresholds. If signal file 105 does not pass the signal analysis, then analytical engine 120 may reject signal file 105 and/or request a new signal file 105 from provider 101 at 320. Else, method 300 may continue at 325.

At 325, the signal file is provided to one or more add-on modules. Signal file 105 may be provided to one or more add-on modules 130 by analytical engine 120. Each add-on module 130 may be associated with a different condition, such as a medical condition or disease. Depending on the embodiment, signal file 105 may be provided to every add-on module 130 of evaluation system 100, to only those add-on modules 130 that correspond to a condition that was selected and/or paid for by the provider 101 that submitted signal file 105, or to other combinations of add-on modules as may be desirable.

In some embodiments, signal analysis engine 225 may further generate one or more visualizations of the signals in signal file 105 after the SQA is complete. The visualizations may be stored by signal analysis engine 225 with signal file 105.

At 330, a score is received from each of the one or more add-on modules. Scores 135 may be received by report engine 140 of evaluation system 100 from each of the one or more add-on modules 130. In some embodiments, scores 135 may be stored in the account associated with provider 101 and/or patient.

At 335, a report is generated. Report 145 may be generated in part by report engine 140 of medical evaluation system 100 and one or more of the add-on modules 130. Report 145 may include each received score 135 along with context and statical information about the received score. Report 145 may further include information related to the acquired biophysical information of the patient. The contents of report 145 related to a received score 135 may be generated by its corresponding add-on module 130. The contents of report 145 related to the acquired biophysical information may be generated by report engine 140, for example.

At 340, the report is provided. Report 145 may be provided to provider 101 through a web-based portal or software application as described above, viewable on a computer, tablet, mobile device, etc. In other embodiments, report 145 may be made available via the user interface on the signal capture device 102. Report 145 may also be directly sent to an electronic medical records system associated with the patient and/or provider 101. In some embodiments, evaluation system 100 may send a message (e.g., e-mail, text message, visual and/or audible alert, etc.) to provider 101 or any number of other entities or individuals indicating that report 145 is available. Provider 101 may then, for example, log into evaluation system 100 to view the report 145 using the portal on a personal computer, tablet computer, mobile device (mobile phone, watch, etc.), or other devices, for example.

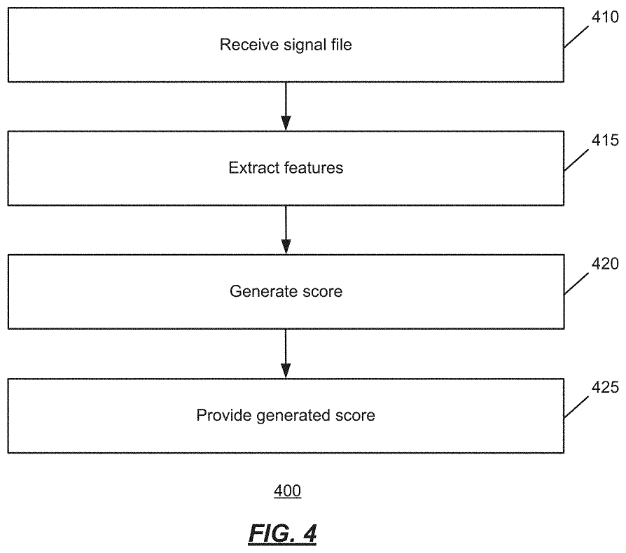

FIG. 4 is an illustration of a method 400 for generating a score by an add-on module 130 of evaluation system 100.

At 410, a signal file is received. Signal file 105 may be received by add-on module 130 from analytical engine 120. Signal file 105 may be associated with a patient and may include biophysical signals acquired from the patient by a signal recorder or capture device 102. Signal file 105 may have passed an SQA performed by signal analysis engine 225. Depending on the embodiment, signal file 105 may have been placed in a signal queue 235 corresponding to add-on module 130 (or all of the add-on modules 130), and add-on module 130 may retrieve signal file 105 from signal queue 235. Add-on module 130 may also receive (or retrieve) patient data 107 corresponding to the patient associated with signal file 105.

At 415, features are extracted from the signal file. Features 243 may be extracted from signal file 105 by feature extraction engine 240 of add-on module 130. Depending on the embodiment, one or more features 243 may also be extracted from patient data 107. Extracted features 243 may include those features 243 that are used by model 260 associated with add-on module 130. Extracted features 243 may be features 243 that have been determined to be relevant to assessing the status of the patient's health in the context of the medical condition associated with the add-on module 130 for the patient, and in some embodiments predicting whether or assessing the probability that, a patient will develop such a medical condition.

At 420, a score is generated. Score 135 may be generated by assessment engine 250 using model 260 and extracted features 243. Score 135 may represent the probability or likelihood that the patient associated with signal file 105 either has and/or in some embodiments will develop the medical condition associated with add-on module 130.

At 425, the generated score is provided. Generated score 135 may be provided by assessment engine 250 to report engine 140, where it may be added to the report 145 that is provided to the provider 101.

FIG. 5 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, wearable devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc., that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 5, an exemplary system for implementing aspects described herein includes a computing device, such as computing device 500. In its most basic configuration, computing device 500 typically includes at least one processing unit 502 and memory 504. Depending on the exact configuration and type of computing device, memory 404 may be volatile (such as random access memory (RAM)), non-volatile (such as read-only memory (ROM), flash memory, etc.), or some combination of the two. This most basic configuration is illustrated in FIG. 5 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 5 by removable storage 508 and non-removable storage 510.

Computing device 500 typically includes a variety of computer-readable media. Computer-readable media can be any available media that can be accessed by the device 500 and includes both volatile and non-volatile media, removable and non-removable media.

Computer storage media include volatile and non-volatile, and removable and non-removable media implemented in any method or technology for storage of information such as computer-readable instructions, data structures, program modules, or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device such as a microphone, biometric sensor, touch input device, camera, etc. Output device(s) 516 such as a display, speakers, printer, etc. may also be included. All these devices are well known in the art and need not be discussed at length here.

It should be understood that the various techniques described herein may be implemented in connection with hardware components and/or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, solid state thumb drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Additional description of the various features are described in U.S. Provisional Patent Application No. 63/235,960, filed Aug. 23, 2021, entitled “Method and System to Non-Invasively Assess Elevated Left Ventricular End-Diastolic Pressure”; U.S. Provisional Patent Application No. 63/236,072, filed Aug. 23, 2021, entitled “Methods and Systems for Engineering Visual Features From Biophysical Signals for Use in Characterizing Physiological Systems”; U.S. Provisional Patent Application No. 63/235,963, filed Aug. 23, 2021, entitled “Methods and Systems for Engineering Power Spectral Features From Biophysical Signals for Use in Characterizing Physiological Systems”; U.S. Provisional Patent Application No. 63/235,966, filed Aug. 23, 2021, entitled “Methods and Systems for Engineering Respiration Rate-Related Features From Biophysical Signals for Use in Characterizing Physiological Systems”; U.S. Provisional Patent Application no. 63/235,968, filed Aug. 23, 2021, entitled “Methods and Systems for Engineering Wavelet-Based Features From Biophysical Signals for Use in Characterizing Physiological Systems”; U.S. patent application Ser. No. 17/558,702, entitled “Method and System to Assess Disease Using Cycle Variability Analysis of Cardiac and Photoplethysmographic Signals”; U.S. Provisional Patent Application No. 63/235,971, filed Aug. 23, 2021, entitled “Methods and Systems for Engineering photoplethysmographic Waveform Features for Use in Characterizing Physiological Systems”; U.S. Provisional Patent Application No. 63/236,193, filed Aug. 23, 2021, entitled “Method and System to Assess Disease Using Morphological Analysis of Cardiac Signals”; U.S. Provisional Patent Application No. 63/235,974, filed Aug. 23, 2021, entitled “Methods and Systems for Engineering Conduction Deviation Features From Biophysical Signals for Use in Characterizing Physiological Systems,”, each of which is hereby incorporated by reference herein in its entirety.

Further examples of processing that may be used with the exemplified method and system disclosed herein (e.g., as features) are described in: U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Patent Publication nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication nos. WO2017/033164; WO2017/221221; WO2019/130272; WO2018/158749; WO2019/077414; WO2019/130273; WO2019/244043; WO2020/136569; WO2019/234587; WO2020/136570;WO2020/136571; U.S. patent application Nos. 16/831,264; 16/831,380; 17/132869; PCT Application nos. PCT/IB2020/052889; PCT/IB2020/052890, each of which has been incorporated by reference herein in its entirety.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A medical evaluation system comprising:
a base system; and
one or more add-on modules, wherein each add-on module is associated with a different condition of a plurality of conditions;
wherein the base system is adapted to conduct one or more operations, comprising:
receive a signal file that comprises a plurality of signals obtained from a patient;
provide the signal file to at least one add-on module of the one or more add-on modules;
receive a score from the at least one add-on module, wherein the score relates to the health of the patient;
generate a report including the received score; and
provide the generated report.

2. The medical evaluation system of claim 1, wherein the base system comprises an analytical engine.

3. The medical evaluation system of claim 2, wherein the analytical engine analyzes the signal file for quality and other metrics.

4. The medical evaluation system of claim 1, wherein the score represents a probability or likelihood that the patient has a condition associated with the at least one add-on module.

5. The medical evaluation system of claim 1, wherein each add-on module is adapted to:
receive the signal file from the base system;
extract one or more of features from the signal file;
generate the score using at least one of the features of the extracted one or more features and a model trained to generate the score; and
provide the generated score to the base system.

6. The medical evaluation system of claim 5, wherein the model comprises one or more of a linear model, a decision tree model, a random forest model, a support vector machine model, a neural network model, a deep neural network model, a convolutional neural network model, an adversarial neural network model, and a genetic algorithm model.

7. The system of claim 1, wherein the plurality of signals comprises biophysical signals.

8. The medical evaluation system of claim 7, wherein the plurality of signals comprises one or more of ballistic signals and acoustic signals.

9. The medical evaluation system of claim 7, wherein the biophysical signals comprise one or more biopotential signals and one or more photoplethysmographic signals.

10. The medical evaluation system of claim 9, wherein the biophysical signals comprise one or more of ballistic signals and acoustic signals.

11. The medical evaluation system of claim 1, wherein the base system is further adapted to:
perform a quality assessment of the plurality of signals; and
request a new signal file if the plurality of signals fail the quality assessment.

12. The medical evaluation system of claim 1, wherein the signal file is received from a medical device.

13. The medical evaluation system of claim 1, wherein the base system is part of a cloud-computing device.

14. The medical evaluation system of claim 1, wherein the base system is approved for marketing by a regulatory authority, and at least one of the add-on modules is received after the system was cleared for marketing by a regulatory agency or body.

15. The medical evaluation system of claim 1, wherein the base system is further adapted to:
provide the signal file to each add-on module of a subset of add on modules;
receive a score from each add-on module; and
generate the report including each received score.

16. The medical evaluation system of claim 1, wherein the base system is further adapted to:
receive a selection of a subset of the one or more add-on modules;
provide the signal file to each add-on module of the subset;
receive a score from each add-on module of the subset; and
generate the report including each received score.

17. The medical evaluation system of claim 16, wherein the base system is further adapted to charge a fee to a user thereof to obtain the score for each add-on module in the subset.

18. The medical evaluation systems of claim 1, wherein the base system conducts the operations without any change to any software, firmware, or documentation thereto.

19. A method comprising:
receiving a signal file by a computing system from a device, wherein the signal file comprises a plurality of signals obtained from a patient using a plurality of sensors associated with the device;
extracting one or more features from the signal file by the computing system;
selecting a model trained to output a score with respect to a condition using at least some of the features of the plurality of features;
generating a score using at least some of the features of the extracted one or more features by the computing system, wherein the score relates to the health of the patient ; and
providing the generated score to the device by the computing system.

20. A medical evaluation system comprising:
a signal capture device adapted to:
capture a plurality of signals from a patient; and
generate a signal file that includes the plurality of signals;
a base system; and
one or more add-on modules, wherein each add-on module is associated with a different condition of a plurality of conditions, and wherein the base system is adapted to conduct one or more operations comprising:
perform a quality assessment of the plurality of signals of the signal file;
provide the signal file to at least one add-on module of the one or more add-on modules;
receive a score from the at least one add-on module, wherein the score relates to the health of the patient;
generate a report including the received score and information about the signal file; and
provide the generated report.
